(19) United States
(12) Patent Application Publication
(10) Pub. No.: US 2024/0182959 A1
(43) Pub. Date: Jun. 6, 2024

(54) REVERSIBLY CROSS-LINKED HYDROGELS, AND METHODS OF USING THE SAME FOR CLUSTER AMPLIFICATION

(71) Applicant: Illumina, Inc., San Diego, CA (US)

(72) Inventors: Hassan Bohra, Singapore (SG); Johan Basuki, Singapore (SG); Wei Wei, Singapore (SG); Brian Mather, San Diego, CA (US)

(73) Assignee: Illumina, Inc., San Diego, CA (US)

(21) Appl. No.: 18/515,013

(22) Filed: Nov. 20, 2023

Related U.S. Application Data
(60) Provisional application No. 63/427,018, filed on Nov. 21, 2022.

(51) Int. Cl. C12Q 1/6855; B01L 3/00; C12Q 1/6806

(52) U.S. Cl. CPC C12Q 1/6855; B01L 3/508; C12Q 1/6806; B01L 2200/0647; B01L 2200/16; B01L 2300/0636; B01L 2300/069; B01L 2300/0809

(57) ABSTRACT

Some examples herein provide a hydrogel on a substrate. The hydrogel includes a three-dimensional network of polymer chains; first functional groups coupled to the polymer chains; amplification primers coupled to the polymer chains via the first functional groups; and second functional groups coupled to the polymer chains and reversibly cross-linking the polymer chains to one another. Some examples herein provide a method of using a hydrogel. The method includes hybridizing a target polynucleotide to an amplification primer coupled to a hydrogel; cleaving cross-linkages within the hydrogel within which the target polynucleotide is hybridized to the amplification primer; and amplifying the target polynucleotide using additional amplification primers within the hydrogel within which the cross-linkages have been cleaved.

### CROSS-REFERENCE TO RELATED APPLICATION

This application claims the benefit of U.S. Provisional Patent Application No. 63/427,018 filed Nov. 21, 2022, and entitled “Reversibly Cross-Linked Hydrogels, and Methods of Using the Same for Cluster Amplification,” the disclosure of which is hereby incorporated by reference in its entirety.

### FIELD

This application relates to hydrogels, such as may be used in cluster amplification.

### BACKGROUND

Cluster amplification is an approach to amplifying polynucleotides, for example for use in genetic sequencing. Target polynucleotides are captured by primers (e.g., P5 and P7 primers) coupled to a substrate surface in a flowcell, and form “seeds” at random locations on the surface. Cycles of amplification are performed to form clusters on the surface around each seed. The clusters include copies, and complementary copies, of the seed polynucleotides. In some circumstances, the substrate is patterned so as to define regions that bound different clusters, such as wells that may be filled with respective clusters.

### SUMMARY

Examples provided herein are related to reversibly cross-linked hydrogels, and methods of using the same for cluster amplification.

Some examples herein provide a hydrogel on a substrate. The hydrogel may include a three-dimensional network of polymer chains. The hydrogel may include first functional groups coupled to the polymer chains. The hydrogel may include amplification primers coupled to the polymer chains via the first functional groups. The hydrogel may include second functional groups coupled to the polymer chains and reversibly cross-linking the polymer chains to one another.

In some examples, the first and second functional groups are of different types than one another. In other examples, the first and second functional groups are of the same type as one another. In some examples, the first and second functional groups independently are selected from the group consisting of: azide, amine, thiol, diol, aldehyde, alkyne, strained cyclooctyne, and an inverse electron-demand (IED) Diels-Alder group.

In some examples, the second functional groups reversibly cross-link the polymer chains via cleavable molecules. In some examples, the cleavable molecules are cleavable using a chemical agent, an enzyme, light, or heat. In some examples, the chemical agent includes an acid. In some examples, the cleavable molecules include an acetal, ketal, imine, hydrazone, or t-butyl ester that is cleavable by the acid. In other examples, the chemical agent includes a reducing agent. In some examples, the cleavable molecules include a disulfide bond or azidoalkyl ether that is cleavable using the reducing agent, or allyl ether that is cleavable using a palladium complex of the reducing agent. In other examples, the enzyme includes a DNAase, RNAase, protease, or restriction enzyme, and wherein the cleavable molecules include an oligonucleotide that is cleavable using the DNAase, RNAase, protease, or restriction enzyme. In other examples, the enzyme includes a protease enzyme or lysosomal enzyme, and wherein the cleavable molecules include a peptide that is cleavable using the protease enzyme or lysosomal enzyme. In other examples, the cleavable molecules include a Diels-Alder conjugation that is cleavable using heat. In other examples, the cleavable molecules include a coumarin or nitrobenzene group that is cleavable using light.

In some examples, the second functional groups include host molecules that reversibly cross-link the backbone via guest molecules. In some examples, the guest molecules are removable via salt, heat, or pH. Additionally, or alternatively, in some examples, the guest molecules are removable via displacement with a binding partner to the guest molecules. Additionally, or alternatively, in some examples the host molecules include crown ethers and the guest molecules include ammonium moieties. Additionally, or alternatively, host molecules include beta-cyclodextrins and the guest molecules include adamantanes, ferrocenes, or bipyridines. In some examples, the second functional groups include ligand molecules that reversibly cross-link the backbone via multivalent binding proteins. In some examples, the multivalent binding proteins are removable using a denaturing agent.

Some examples herein provide a method of using a hydrogel. The method may include depositing a hydrogel on a substrate. The hydrogel may include a three-dimensional network of polymer chains and at least first and second types of functional groups coupled to the polymer chains. The method may include coupling amplification primers to the first functional groups of the deposited hydrogel. The method may include reversibly stabilizing the deposited hydrogel by reversibly cross-linking the second functional groups of the deposited hydrogel to which the amplification primers are coupled.

Some examples herein provide another method of using a hydrogel. The method may include depositing a hydrogel on a substrate. The hydrogel may include a three-dimensional network of polymer chains, amplification primers coupled to the polymer chains, and functional groups coupled to the polymer chains. The method may include reversibly stabilizing the hydrogel by reversibly cross-linking the functional groups of the deposited hydrogel to which the amplification primers are coupled.

Some examples herein provide another method of using a hydrogel. The method may include depositing a hydrogel on a substrate, the hydrogel including three-dimensional network of polymer chains and first functional groups coupled to the polymer chains. The method may include coupling amplification primers to a first subset of the first functional groups of the deposited hydrogel. The method may include converting a second subset of the first functional groups to second functional groups. The method may include reversibly stabilizing the hydrogel by reversibly cross-linking the second functional groups.

Some examples herein provide another method of using a hydrogel. The method may include hybridizing a target polynucleotide to an amplification primer coupled to a hydrogel. The method may include cleaving cross-linkages within the hydrogel within which the target polynucleotide is hybridized to the amplification primer. The method may include amplifying the target polynucleotide using additional amplification primers within the hydrogel within which the cross-linkages have been cleaved. Optionally, the method further may include swelling the hydrogel after the cleaving and before the amplifying.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIGS. 1A-1F schematically illustrate example compositions and operations in a process flow for using reversibly cross-linked hydrogels for cluster amplification.

FIGS. 2A-2C schematically illustrate alternative example compositions and operations in a process flow for using reversibly cross-linked hydrogels for cluster amplification.

FIG. 3 schematically illustrates another alternative example composition and operation in a process flow for using reversibly cross-linked hydrogels for cluster amplification.

FIG. 4 schematically illustrate example hydrogels and example cross-linkers that may be used with such hydrogels.

FIGS. 5A-5B schematically illustrate additional example hydrogels and example cross-linkers that may be used with such hydrogels.

FIG. 6 schematically illustrates an example cleavable molecule that may be used to reversibly cross-link hydrogels in a manner such as described herein.

FIG. 7 schematically illustrates additional example cleavable molecules that may be used to reversibly cross-link hydrogels in a manner such as described herein.

FIGS. 8A-8B schematically illustrate additional example compositions and operations in a process flow for using reversibly cross-linked hydrogels for cluster amplification.

FIGS. 9A-9B schematically illustrate additional example compositions and operations in a process flow for using reversibly cross-linked hydrogels for cluster amplification.

### DETAILED DESCRIPTION

Examples provided herein are related to reversibly cross-linked hydrogels, and methods of using the same for cluster amplification.

It may be desirable to perform sequencing-by-synthesis (SBS) using functionalized hydrogels within flowcells, to determine the sequence of target polynucleotides in respective clusters. For example, a hydrogel may include functional groups to which amplification primers may be coupled. The hydrogel then may be seeded by flowing through the flowcell a target polynucleotide to which has been coupled an adapter that is complementary to the amplification primers, and that hybridizes to one of the primers. The seed then may be amplified using amplification primers that are within the hydrogel, resulting in a cluster including a plurality of amplicons of the target polynucleotide that then may be sequenced using SBS.

However, certain previously known hydrogels may degrade during shipping or storage, which may cause their performance to deteriorate. For example, during shipping or storage, the hydrogel may irreversibly collapse. As such, amplification primers within the hydrogel may be inaccessible during amplification, resulting in generation of clusters that include fewer amplicons and therefore generate less signal during SBS than would clusters formed using a hydrogel that was not so degraded. This can reduce accuracy of the sequencing read.

The reversibly cross-linked hydrogels provided herein are expected to be significantly more stable during shipping and storage than previously known hydrogels such as described above. More specifically, the present hydrogels may be cross-linked prior to shipping and storage. The cross-linkages may reduce or inhibit irreversible collapse of the hydrogel during shipping and storage, for example by limiting polymer chain mobility to form strong inter-chain physical bonds and/or intra-chain physical bonds, in other words, by limiting polymer chain mobility to rearrange into a lower entropy state that would be energetically unfavorable to reverse upon rehydration. The cross-linkages may be controllably reversed, e.g., cleaved or removed by applying an appropriate stimulus, following which the hydrogel may be used as desired.

Additionally, in some examples, the seeding process may be performed before reversing the cross-linkages. As such, amplification primers within the hydrogel may be relatively inaccessible during the seeding process, while amplification primers at the exposed surface of the hydrogel may be used for seeding. In some examples, the cross-linking then may be reversed, making the amplification primers within the hydrogel accessible for amplification. Using the present reversible cross-linking to reduce the number of amplification primers that are available for seeding may reduce the number (or density) of seeding events that occur and thus may improve monoclonality of the clusters that are later formed than would a hydrogel that was not cross-linked during seeding. Additionally, using the present reversible cross-linking to subsequently increase the number of amplification primers that are available for amplification may generate more amplicons than would a hydrogel that had irreversibly collapsed because it had not been cross-linked. As such, it may be understood that the present reversibly cross-linked hydrogels may be expected to provide clusters with enhanced monoclonality and enhanced amplification as compared to previously known hydrogels. However, it will be appreciated that the cross-linking may be used at any suitable time, e.g., may be used to improve hydrogel stability during shipping and storage and may be reversed prior to seeding, and need not necessarily be used to modulate accessibility of amplification primers during seeding and/or amplification.

First, some terms used herein will be briefly explained. Then, some example methods for reversibly cross-linking hydrogels, resulting compositions, and methods of using reversibly cross-linked hydrogels will be described.

Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, “hybridize” is intended to mean noncovalently associating a first polynucleotide to a second polynucleotide along the lengths of those polynucleotides to form a double-stranded “duplex.” For instance, two DNA polynucleotide strands may associate through complementary base pairing. The strength of the association between the first and second polynucleotides increases with the complementarity between the sequences of nucleotides within those polynucleotides. The strength of hybridization between polynucleotides may be characterized by a temperature of melting (Tm) at which 50% of the duplexes have polynucleotide strands that disassociate from one another.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. Examples of polynucleotides include deoxyribonucleic acid (DNA), ribonucleic acid (RNA), and analogues thereof. A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primed single stranded target polynucleotide, and can sequentially add nucleotides to the growing primer to form a “complementary copy” polynucleotide having a sequence that is complementary to that of the target polynucleotide. Another polymerase, or the same polymerase, then can form a copy of the target nucleotide by forming a complementary copy of that complementary copy polynucleotide. Any of such copies may be referred to herein as “amplicons.” DNA polymerases may bind to the target polynucleotide and then move down the target polynucleotide sequentially adding nucleotides to the free hydroxyl group at the 3′ end of a growing polynucleotide strand (growing amplicon). DNA polymerases may synthesize complementary DNA molecules from DNA templates and RNA polymerases may synthesize RNA molecules from DNA templates (transcription). Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase. Example polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity.

As used herein, the term “primer” is defined as a polynucleotide to which nucleotides may be added via a free 3′ OH group. A primer may include a 3′ block preventing polymerization until the block is removed. A primer may include a modification at the 5′ terminus to allow a coupling reaction or to couple the primer to another moiety. A primer may include one or more moieties which may be cleaved under suitable conditions, such as UV light, chemistry, enzyme, or the like. The primer length may be any suitable number of bases long and may include any suitable combination of natural and non-natural nucleotides. A target polynucleotide may include an “adapter” that hybridizes to (has a sequence that is complementary to) a primer, and may be amplified so as to generate a complementary copy polynucleotide by adding nucleotides to the free 3′ OH group of the primer. An “amplification primer” is intended to mean a primer that is coupled to the substrate and may hybridize to a second adapter of the target polynucleotide, while an “orthogonal amplification primer” is intended to mean a primer that is coupled to the substrate and may hybridize to a first adapter of that target polynucleotide. The first adapter may have a sequence that is complementary to that of the orthogonal amplification primer, and the second adapter may have a sequence that is complementary to that of the amplification primer. An amplification primer and an orthogonal amplification primer may have different and independent sequences than one another. In one nonlimiting example, the amplification primer includes a P5 primer, and the orthogonal amplification primer includes a P7 primer.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, substrates may include silicon, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface comprising glass or a silicon-based polymer. In some examples, the substrates may include a metal. In some such examples, the metal is gold. In some examples, the substrate has at least one surface comprising a metal oxide. In one example, the surface comprises a tantalum oxide or tin oxide. Acrylamides, enones, or acrylates may also be utilized as a substrate material or component. Other substrate materials may include, but are not limited to gallium arsenide, indium phosphide, aluminum, ceramics, polyimide, quartz, resins, polymers and copolymers. In some examples, the substrate and/or the substrate surface may be, or include, quartz. In some other examples, the substrate and/or the substrate surface may be, or include, semiconductor, such as GaAs or ITO. The foregoing lists are intended to be illustrative of, but not limiting to the present application. Substrates may comprise a single material or a plurality of different materials. Substrates may be composites or laminates. In some examples, the substrate comprises an organo-silicate material. Substrates may be flat, round, spherical, rod-shaped, or any other suitable shape. Substrates may be rigid or flexible. In some examples, a substrate is a bead or a flow cell.

In some examples, a substrate includes a patterned surface. A “patterned surface” refers to an arrangement of different regions in or on an exposed layer of a substrate. For example, one or more of the regions may be features where one or more amplification primers are present, for example in a hydrogel located within that feature. The features can be separated by interstitial regions where amplification primers and hydrogel are not present. In some examples, the pattern may be an x-y format of features that are in rows and columns. In some examples, the pattern may be a repeating arrangement of features and/or interstitial regions. In some examples, the pattern may be a random arrangement of features and/or interstitial regions. In some examples, a substrate includes an array of wells (depressions) in a surface. The wells may be provided by substantially vertical sidewalls. Wells may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The features in a patterned surface of a substrate may include wells in an array of wells (e.g., microwells or nanowells) on glass, silicon, plastic or other suitable material(s) with patterned hydrogel such as provided herein. The patterning may provide hydrogel pads that may be used for sequencing, e.g., may be stable over sequencing runs with a large number of cycles. In some examples, covalent linking of the hydrogel to the wells may be helpful for maintaining the hydrogel in the structured features (e.g., wells) throughout the lifetime of the structured substrate during a variety of uses. However in some examples, the hydrogel need not be covalently linked to the wells.

In particular examples, a structured substrate may be made by patterning a substrate formed of suitable material with wells (e.g. microwells or nanowells), coating the substrate material with a hydrogel, and polishing the surface of the hydrogel coated material, for example via chemical or mechanical polishing, thereby retaining hydrogel in the wells but removing or inactivating substantially all of the hydrogel from the interstitial regions on the surface of the structured substrate between the wells. Amplification primers may be attached to the hydrogel, e.g., in a manner such as provided herein. A solution including a plurality of target polynucleotides (e.g., a fragmented human genome or portion thereof) may then be contacted with the polished substrate such that individual target polynucleotides will seed individual wells via interactions with amplification primers attached to the hydrogel; however, the target polynucleotides will not occupy the interstitial regions due to absence or inactivity there of the hydrogel. Amplification of the target polynucleotides may be confined to the wells because absence or inactivity of hydrogel in the interstitial regions may inhibit outward migration of the growing cluster. The process is conveniently manufacturable, being scalable and utilizing conventional micro- or nano-fabrication methods.

A patterned substrate may include, for example, wells etched into a slide or chip. The pattern of the etchings and geometry of the wells may take on a variety of different shapes and sizes, and such features may be physically or functionally separable from each other. Particularly useful substrates having such structural features include patterned substrates that may select the size of solid particles such as microspheres. An example patterned substrate having these characteristics is the etched substrate used in connection with BEAD ARRAY technology (Illumina, Inc., San Diego, Calif.).

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that may be used in methods and compositions set forth herein include, but are not limited to, those commercially available from Illumina, Inc. (San Diego, CA).

As used herein, the term “directly” when used in reference to a layer covering the surface of a substrate is intended to mean that the layer covers the substrate's surface without a significant intermediate layer, such as, e.g., an adhesive layer or a polymer layer. Layers directly covering a surface may be attached to this surface through any chemical or physical interaction, including covalent bonds or non-covalent adhesion.

As used herein, the term “immobilized” when used in reference to a polynucleotide is intended to mean direct or indirect attachment to a substrate via covalent or non-covalent bond(s). In certain examples, covalent attachment may be used, or any other suitable attachment in which the polynucleotides remain stationary or attached to a substrate under conditions in which it is intended to use the substrate, for example, in polynucleotide amplification or sequencing. Polynucleotides to be used as amplification primers or as target polynucleotides may be immobilized such that a 3′-end is available for enzymatic extension and at least a portion of the sequence is capable of hybridizing to a complementary sequence. Immobilization may occur via hybridization to a surface attached oligonucleotide, in which case the immobilized oligonucleotide or polynucleotide may be in the 3′-5′ orientation. Alternatively, immobilization may occur by means other than base-pairing hybridization, such as covalent attachment.

As used herein, the term “array” refers to a population of substrate regions that may be differentiated from each other according to relative location. Different molecules (such as polynucleotides) that are at different regions of an array may be differentiated from each other according to the locations of the regions in the array. An individual region of an array may include one or more molecules of a particular type. For example, a substrate region may include a single target polynucleotide having a particular sequence, or a substrate region may include several polynucleotides having the same sequence (or complementary sequences thereof). The regions of an array respectively may include different features than one another on the same substrate. Example features include without limitation, wells in a substrate, beads (or other particles) in or on a substrate, projections from a substrate, ridges on a substrate or channels in a substrate. The regions of an array respectively may include different regions on different substrates than each other. Different molecules attached to separate substrates may be identified according to the locations of the substrates on a surface to which the substrates are associated or according to the locations of the substrates in a liquid or gel. Example arrays in which separate substrates are located on a surface include, without limitation, those having beads in wells.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Example polynucleotide pluralities include, for example, populations of about 1×105 or more, 5×105 or more, or 1×106 or more different polynucleotides. Accordingly, the definition of the term is intended to include all integer values greater than two. An upper limit of a plurality may be set, for example, by the theoretical diversity of polynucleotide sequences in a sample.

As used herein, the term “double-stranded,” when used in reference to a polynucleotide, is intended to mean that all or substantially all of the nucleotides in the polynucleotide are hydrogen bonded to respective nucleotides in a complementary polynucleotide.

As used herein, the term “single-stranded,” when used in reference to a polynucleotide, means that essentially none of the nucleotides in the polynucleotide are hydrogen bonded to a respective nucleotide in a complementary polynucleotide.

As used herein, the term “target polynucleotide” is intended to mean a polynucleotide that is the object of an analysis or action. The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an adapter that functions as a primer binding site, that flank(s) a target polynucleotide sequence that is to be analyzed. A target polynucleotide hybridized to an amplification primer may include nucleotides that extend beyond the 5′ or 3′ end of the amplification primer in such a way that not all of the target polynucleotide is amenable to extension. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an adapter at a single end, and such adapter may be located at either the 3′ end or the 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “amplicon,” when used in reference to a polynucleotide, is intended to means a product of copying the polynucleotide, wherein the product has a nucleotide sequence that is substantially the same as, or is substantially complementary to, at least a portion of the nucleotide sequence of the polynucleotide. “Amplification” and “amplifying” refer to the process of making an amplicon of a polynucleotide. A first amplicon of a target polynucleotide may be a complementary copy. Additional amplicons are copies that are created, after generation of the first amplicon, from the target polynucleotide or from the first amplicon. A subsequent amplicon may have a sequence that is substantially complementary to the target polynucleotide or is substantially identical to the target polynucleotide. It will be understood that a small number of mutations (e.g., due to amplification artifacts) of a polynucleotide may occur when generating an amplicon of that polynucleotide.

As used herein, a “hydrogel” refers to a three-dimensional polymer network structure that includes polymer chains and is at least partially hydrophilic and contains water within spaces between the polymer chains. A hydrogel may include any suitable combination of hydrophilic, hydrophobic, and/or amphiphilic polymer(s), so long as the overall polymer network is hydrophilic and contains water within spaces between the polymer chains. Hydrogels include chemical hydrogels in which both the bonding to form the polymer chains, and any cross-linking between the polymer chains, is covalent; such cross-linking during hydrogel formation may be irreversible, as distinguished from the present reversible cross-linking which is performed after the hydrogel is formed. In some cases, the chemical hydrogel may include, or may consist essentially of, brush-like structures of polymer chains attached to a surface, substantially without physical or covalent crosslinks between polymer chains, or alternatively polymer chains with multiple attachment points to a surface, resulting in loops, but also lacking interchain crosslinks. Hydrogels also include physical hydrogels in which the bonding to form the polymer chains, and any cross-linking within the polymer chains, is not covalent. Nonlimiting examples of physical hydrogels include agarose and alginate.

As used herein, the “polymer chain” of a hydrogel is intended to mean those portions of the hydrogel that are polymerized with one another during the polymerization process. Polymer chains may be cross-linked to form the hydrogel. For example, cross-linkers may be added during or after the polymerization process that forms the polymer chains. Additionally, or alternatively, in some examples the polymer chains may be deposited on a substrate surface that includes functional groups to which functional groups of the polymer chains become coupled. The polymer chains may be coupled to the surface, e.g., via reactions between the functional groups of the polymer chains and the functional groups at the surface, and such coupling may cross-link the polymer chains to form the hydrogel. Such cross-linking may cause the polymer chains to covalently or non-covalently attach to one another, or may occur as a result of chain entanglement during polymerization and/or attachment to a surface. As provided herein, polymer chains also or alternatively may be reversibly cross-linked after the hydrogel is formed, e.g., using functional groups in a manner such as described in greater detail below.

As used herein, the term “functional group” is intended to mean a chemical entity that is reactive with another chemical entity of the same type, or of a different type, to form a covalent bond or a non-covalent bond. As provided herein, when a hydrogel is described as having a functional group, it is to be understood that the functional group is chemically different from the polymer chains. Accordingly, the functional groups may not necessarily participate in forming the three-dimensional network of polymer chains during formation of the hydrogel or attachment of the polymer chains to a surface; as such, and as provided herein, the functional groups are available to perform additional cross-linking of the hydrogel at a time after the hydrogel itself is formed and is disposed on a substrate.

Methods of forming and using reversibly cross-linked hydrogels

As provided herein, stability of a hydrogel may be enhanced by reversibly cross-linking the hydrogel, e.g., after hydrogel formation (after formation and cross-linking of the polymer chains to form the hydrogel is complete) and before hydrogel shipping and/or storage. Additionally, or alternatively, as provided herein, the hydrogel optionally may be reversibly cross-linked at any suitable time before seeding the hydrogel with a template polynucleotide, and the cross-linkages optionally may be reversed at any suitable time before or during conducting cluster amplification using the seeded template polynucleotide. Alternatively, the cross-linkages optionally may be reversed before seeding.

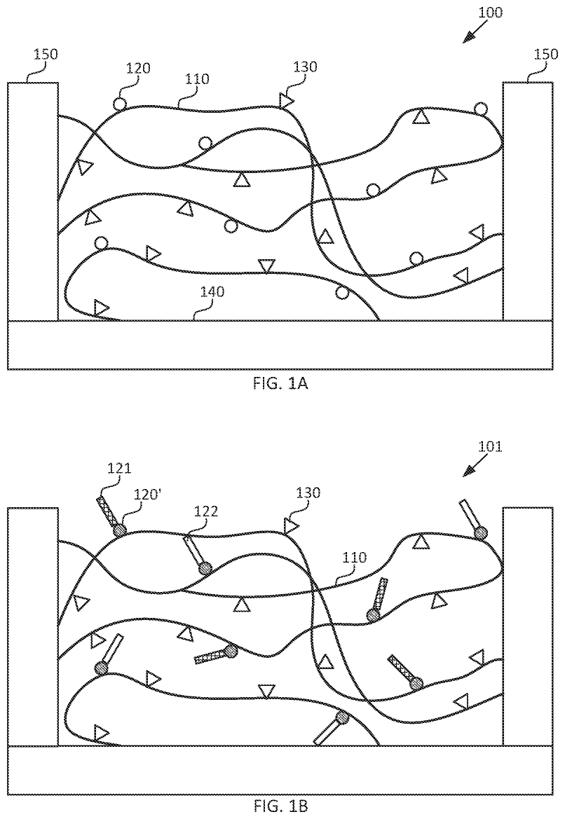

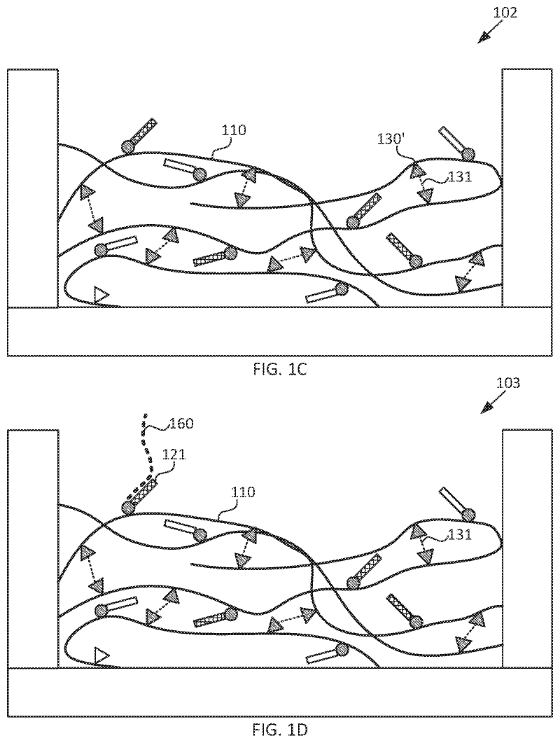

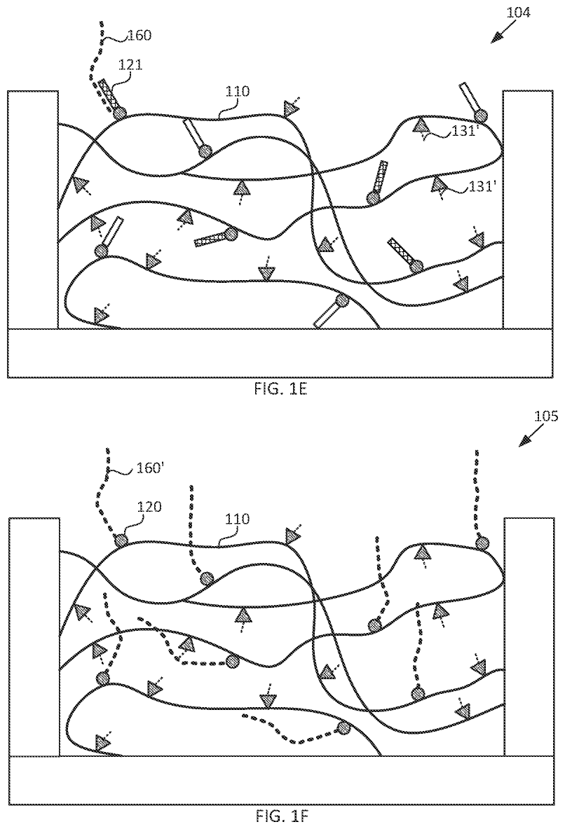

For example, FIGS. 1A-1F schematically illustrate example compositions and operations in a process flow for using reversibly cross-linked hydrogels for cluster amplification. The composition illustrated in FIG. 1A includes hydrogel 100 on substrate 140. Optionally, substrate 140 may include vertical sidewalls 150 that laterally constrain hydrogel 100. Hydrogel 100 includes a three-dimensional network of polymer chains 100, intended to be represented by the elongated lines that intersect each other. Hydrogel 100 also includes first functional groups 120 coupled to the polymer chains 110, and second functional groups 130 coupled to the polymer chains 110. In the nonlimiting example illustrated in FIG. 1A, first functional groups 120 and second functional groups 130 are different types of chemical entities than one another, and are different types of chemical entities than the monomers forming polymer chains 110 of hydrogel 100. First functional groups 120 may be identical to one another, or may be different than one another. Second functional groups 130 may be identical to one another, or may be different than one another. Hydrogel 100 may be formed in any suitable manner so as to form a three-dimensional cross-linked network of polymer chains 110 that include first functional groups 120 and second functional groups 130. Nonlimiting examples of monomers that may be used to form hydrogel 100 are described in greater detail below. In some examples, hydrogel 100 is a chemical hydrogel in which both the bonding and the cross-linking within the polymer chains 110 of the hydrogel is covalent and thus irreversible. First functional groups 120 and second functional groups 130 substantially do not participate in the formation of the polymer chains 110 of hydrogel 100, or in cross-linking of such polymer chains during formation of hydrogel 100. Instead, in a manner such as will be described in greater detail below, first functional groups 120 may be used to couple amplification primers to the polymer chains 110 after formation of the hydrogel 100, and second functional groups 130 may be used to cross-link the polymer chains 110 to one another after the amplification primers are coupled to the polymer chains. The polymer chains 110 and/or substrate 140 may include other functional groups (not specifically shown) via which the polymer chains may be cross-linked to one another and/or to the substrate 140 during formation of hydrogel 100. Such functional groups, and such cross-linkages during hydrogel formation, are to be distinguished from functional groups 130 that are used to reversibly cross-link the hydrogel after the hydrogel is formed, e.g., minutes, hours, days, or longer after the hydrogel is formed.

Hydrogel 100 illustrated in FIG. 1A may be contacted with a fluid that includes amplification primers that include functional groups (not specifically illustrated) that are configured to react with first functional groups 120 to covalently bond the amplification primers to polymer chains 110. During such contact between the fluid and hydrogel 100, amplification primers 121, 122 may diffuse through open spaces within the three-dimensional cross-linked network of the hydrogel, and thus may become covalently coupled to polymer chains 110 at a variety of different lateral locations and a variety of different depths as is intended to be illustrated in FIG. 1B. For example, hydrogel 100 may have a sufficient amount of open space to facilitate diffusion of amplification primers 121, 122 therethrough. Such contact between the fluid and hydrogel 100 described with reference to FIG. 1A forms modified hydrogel 101 illustrated in FIG. 1B, which includes amplification primers 121, 122 coupled to the polymer chains 110 via the first functional groups 120. In FIG. 1B, first functional groups 120 are represented with dark fill and the reference numeral 120′ to represent that the functional groups have reacted with the functional groups of the amplification primers 121, 122. Additionally, in FIG. 1B, amplification primers 121, 122 are illustrated with different fills than one another to represent a nonlimiting example in which different types of amplification primers are coupled to the hydrogel. For example, amplification primer 121 may include a P5 primer, and amplification primer 122 may include an orthogonal amplification primer, such as a P7 primer.

Hydrogel 101 illustrated in FIG. 1B may be contacted with a fluid that includes at least one chemical or enzymatic reagent that is configured to react with second functional groups 130 cause such functional groups to covalently or non-covalently cross-link the polymer chains 110 of hydrogel 101 to one another. During such contact between the fluid and hydrogel 101, at least one chemical or enzymatic reagent may diffuse through open spaces within the three-dimensional cross-linked network of the hydrogel 101, may cross-link polymer chains 110 at a variety of different lateral locations and a variety of different depths as is intended to be illustrated in FIG. 1C. Open space within hydrogel 101 may facilitate diffusion of the at least one chemical or enzymatic reagent therethrough. Such contact forms modified hydrogel 102 illustrated in FIG. 1C in which second functional groups 130 coupled to polymer chains 110 reversibly cross-link the polymer chains to one another. In FIG. 1C, second functional groups 130 are represented with dark fill and the reference numeral 130′ to represent that the functional groups have reacted so as to cross-link the polymer chains. Additionally, in FIG. 1C, the cross-linkages 131 between polymer chains 110 are illustrated using dashed lines to represent that such cross-linkages are reversible, e.g., in a manner such as will be described in greater detail below.

As a result of cross-linking such as illustrated in FIG. 1C, hydrogel 102 may have a percent open space that is significantly less than that of hydrogel 100 and that is significantly less than that of hydrogel 101, as is intended to be illustrated by the closer proximity of polymer chains 110 within FIG. 1C. The reduced open space of hydrogel 102 may reduce or inhibit diffusion of any reagents therethrough as compared to the open space of hydrogels 100 and 101, e.g., in a manner such as described with reference to FIG. 1D. Additionally, the cross-linking 131 within hydrogel 102 may be expected to significantly stabilize hydrogel 102 as compared to hydrogels 100 and 101, for example during storage or shipping. Illustratively, if hydrogels 100 or 101 are shipped or stored under conditions that cause the hydrogel to fully or partially dry out (lose water), the resulting reduction in distance between polymer chains 110 can cause the polymer chains to become irreversibly hydrogen bonded to one another in such a manner that irreversibly collapses the hydrogel, closing a significant portion of the open space in the hydrogel. As such, even if water is added to the collapsed hydrogel, the water may not significantly penetrate into the collapsed hydrogel and the hydrogel may remain in a collapsed state which is, at best, of limited utility for further use. In comparison, the cross-linking 131 within hydrogel 102 may retain polymer chains 110 at a sufficiently spaced distance from one another as to inhibit such irreversible hydrogen bonding, and thus so as to inhibit irreversible collapse of the hydrogel. As such, upon reversing the cross-linking in a manner such as described below, the hydrogel may be swelled back into an open state in which water and reagents again may be diffused into the hydrogel.

Additionally, the reduced open space of hydrogel 102 optionally may be used to improve monoclonality of a cluster that is generated using hydrogel 102, as compared to a cluster generated using hydrogel 101. For example, hydrogel 102 illustrated in FIG. 1C optionally may be contacted with a fluid that includes target polynucleotides 160 that include first and second adapters (not specifically illustrated) that respectively are complementary to amplification primers 121, 122. During such contact between the fluid and hydrogel 102, the reduced open space within the three-dimensional cross-linked network of the hydrogel 102 may reduce or inhibit diffusion of target polynucleotides 160 into hydrogel 102, as compared to diffusion within hydrogel 101. As a result of the reduced open space within hydrogel 102, the number of amplification primers 121, 122 that are available for target polynucleotides 160 to hybridize to may be significantly lower than the total number of amplification primers 121, 122 within hydrogel 102. Indeed, in some examples, the cross-linking 131 within hydrogel 102 reduces the open space within hydrogel 102 to an extent at which target polynucleotides 160 substantially may not diffuse into the hydrogel, and therefore substantially are limited to hybridizing to amplification primers 121, 122 at the surface of the hydrogel. In comparison, if hydrogel 101 were to be contacted with the same fluid that includes target polynucleotides 160, the target polynucleotides would be expected to readily diffuse into the hydrogel and to hybridize to amplification primers 121, 122 at a variety of different lateral locations and a variety of different depths. Because the number of amplification primers available to hybridize with target polynucleotides 160 is significantly reduced in hydrogel 102 as compared to hydrogel 101, the likelihood of having only a single hybridization event (single seeding event) is significantly greater with hydrogel 102 than with hydrogel 101. Accordingly, a cluster generated using hydrogel 102 may be expected to be significantly more likely to be monoclonal than is a cluster generated using hydrogel 101. Illustratively, in the example shown in FIG. 1D, hydrogel 103 includes only a single target polynucleotide 160 hybridized to amplification primer 121 at the surface of the hydrogel, because cross-linking 131 sufficiently reduces the availability of the amplification primers at greater depths within the hydrogel for such hybridization.

In the nonlimiting example shown in FIG. 1D, after target polynucleotide 160 becomes hybridized to amplification primer 121, the cross-linking 131 between second functional groups 130′ may be reversed. For example, hydrogel 103 illustrated in FIG. 1D may be contacted with a fluid that includes at least one chemical or enzymatic reagent that is configured to react with cross-linkages 131 so as to reverse the covalent or non-covalent cross-linkages between the polymer chains 110. During such contact between the fluid and hydrogel 103, at least one chemical or enzymatic reagent may diffuse through open spaces within the three-dimensional cross-linked network of the hydrogel 103, and may reverse the cross-linkages 131 between polymer chains 110 at a variety of different lateral locations and a variety of different depths as is intended to be illustrated in FIG. 1E. Note that at the beginning of contact with such a fluid, hydrogel 103 may be substantially closed by cross-linkages 131 in a manner such as described with reference to FIG. 1D. However, the fluid may contact the surface of hydrogel 103 and may reverse cross-linkages at that surface, which may cause the hydrogel to open at the surface. Such opening may permit the fluid to diffuse slightly into the hydrogel, and there to reverse additional cross linkages which may cause the hydrogel to open slightly below the surface. In such a manner, as the hydrogel is gradually and directionally opened from its surface, the fluid may diffuse still further into the hydrogel, eventually resulting in re-opening of the entire hydrogel. The resulting open space may facilitate diffusion of the at least one chemical or enzymatic reagent therethrough. Such contact forms modified hydrogel 104 illustrated in FIG. 1E in which the cross-linkages using second functional groups 130′ are broken, as intended to be illustrated using the shortened dashed lines and reference numeral 131′.

In some examples, the target polynucleotide 160 hybridized to amplification primer 121 then may be amplified, e.g., using bridge amplification or other cluster generation technique described elsewhere herein or otherwise known in the art. For example, target polynucleotide 160 may be amplified using processes such as known in the art, e.g., using surface-bound polymerase chain reaction (PCR), bridge amplification, or a strand invasion process which may be referred to as ExAmp, forming a cluster of amplicons 160′ illustrated in FIG. 1F. Such amplification process may use chemical and/or enzymatic reagents that diffuse through open spaces within the three-dimensional cross-linked network of the hydrogel 104, resulting in amplicons 160′ coupled to polymer chains 110 at a variety of different lateral locations and a variety of different depths as is intended to be illustrated in FIG. IF. As illustrated in FIG. 1F, the amplicons 160′ may be directly coupled to the polymer chains 110, e.g., via first functional groups 120 which originally coupled primers 121, 122 to the polymer chains.

In some examples, the amount of open space within hydrogel 101 may be similar to the amount of open space within hydrogel 100, e.g., may be within about 20%, or within about 10%, or within about 5%, of the open space within hydrogel 100. Additionally, or alternatively, in some examples, the cross-link density of hydrogel 101 may be similar to the cross-link density of hydrogel 100, e.g., may be within about 20%, or within about 10%, or within about 5%, of the cross-link density within hydrogel 100. Additionally, or alternatively, in some examples, the amount of open space within hydrogel 103 may be similar to the amount of open space within hydrogel 102, e.g., may be within about 20%, or within about 10%, or within about 5%, of the open space within hydrogel 102. Additionally, or alternatively, in some examples, the cross-link density of hydrogel 103 may be similar to the cross-link density of hydrogel 102, e.g., may be within about 20%, or within about 10%, or within about 5%, of the cross-link density within hydrogel 102. Additionally, or alternatively, in some examples, the amount of open space within hydrogel 104 may be similar to the amount of open space within hydrogel 101, e.g., may be within about 20%, or within about 10%, or within about 5%, of the open space within hydrogel 101. Additionally, or alternatively, in some examples, the cross-link density of hydrogel 104 may be similar to the cross-link density of hydrogel 101, e.g., may be within about 20%, or within about 10%, or within about 5%, of the cross-link density within hydrogel 101. Additionally, or alternatively, in some examples, the amount of open space within hydrogel 105 may be similar to the amount of open space within hydrogel 101, e.g., may be within about 20%, or within about 10%, or within about 5%, of the open space within hydrogel 101. Additionally, or alternatively, in some examples, the cross-link density of hydrogel 105 may be similar to the cross-link density of hydrogel 101, e.g., may be within about 20%, or within about 10%, or within about 5%, of the cross-link density within hydrogel 101. Additionally, or alternatively, in some examples, the open space of hydrogel 102 or 103 may be significantly lower than the open space of hydrogel 100, hydrogel 101, hydrogel 104, and/or hydrogel 105, e.g., may be less than about 80%, or less than about 60%, or less than about 40%, or less than about 20% of the open space within hydrogel 100, hydrogel 101, hydrogel 104, and/or hydrogel 105. Additionally, or alternatively, in some examples, the cross-link density of hydrogel 102 or 103 may be significantly higher than the cross-link density of hydrogel 100, hydrogel 101, hydrogel 104, and/or hydrogel 105, e.g., may be about 20% greater, or about 40% greater, or about 60% greater, or about 80% greater, than the cross-link density within hydrogel 100, hydrogel 101, hydrogel 104, and/or hydrogel 105. Additionally, or alternatively, in some examples, hydrogel 100, 101, 104, and/or 105 may have a thickness that is at least ten times greater than the thickness of hydrogel 102 and/or 103. For example, hydrogel 100, 101, 104, and/or 105 may have a thickness of about 80-120 nm, while hydrogel 102 and/or 103 may have a thickness of about 8-12 nm.

The amount of open space and/or cross-link density within a hydrogel may be characterized using mechanical measurements, such as modulus determined from force distance curves in wet atomic force microscopy (AFM). Additionally, or alternatively, the amount of open space and/or cross-link density within a hydrogel can be measured by the mesh size, e.g., using small angle x-ray scattering (SAXS) or small angle neutron scattering (SANS). Additionally, or alternatively, the amount of open space and/or crosslink density within a hydrogel may be characterized using swelling capacity (mass ratio between dry and wet hydrogel), e.g., using a quartz crystal microbalance (QCM) or ellipsometry in the wet state as compared the dry state. Additionally, or alternatively, the amount of open space and/or crosslink density within a hydrogel may be characterized using modulus (E′ from nano-indentation), which correlate with the hardness/softness of the hydrogel due to crosslinking density. For example, using nano-indentation there will be a difference between highly and less cross-linked hydrogel or polymers. Illustratively, one example strategy to assess openness of the hydrogel in its real condition on flowcell surface (e.g., which may be about 80-120 nm thick in the wet state and about 8-12 nm in the dry state), is using a fluorescence probe by hybridization or base incorporation. Ellipsometry also or alternatively may be used to detect swelling. QCM is compatible with thin hydrogel layers for assessing swelling too, but in some examples the hydrogel may be deposited on the QCM sensor instead of directly on the flowcell substrate. For relatively thin film hydrogel (e.g., <100 nm thickness) nanoindentation may be used. QCM may be used to measure the mass change through water adsorption (open vs close) or measuring primers tethered inside the hydrogel.

Additionally, or alternatively, the amount of open space and/or crosslink density within a hydrogel that includes amplification primers that are available to be hybridized to (e.g., hydrogel 101, 102, 103, or 104) may be characterized using the surface charge. For example, the cross-linking may obscure the primers (which are negatively charged), thus inhibiting the primers from contributing to the surface charge. In some examples, the surface charge may be characterized using atomic force microscopy (AFM), e.g., by measuring the interaction between the surface and the AFM tip. Additionally, or alternatively, Kelvin probe force microscopy may be used to characterize the surface charge of the hydrogel. Additionally, or alternatively, electrokinetic analysis may be used to characterize the surface charge of the hydrogel at a solid-liquid interface. Additionally, or alternatively, the amount of open space and/or crosslink density within a hydrogel that includes amplification primers that are available to be hybridized to (e.g., hydrogel 101, 102, 103, or 104) may be characterized using complementary fluorescent oligonucleotide probes to hybridize to the amplification primers within the hydrogel; the fluorescent intensity of hybridized probes can be used to quantify primers on the surface, and in case of bulky probes such as hairpins, fluorescence can be an indication of how accessible surface primers are to templates in a seeding event. Additionally, or alternatively, the amount of open space and/or cross-link density within a hydrogel can be measured by quantifying cleaved bonds using infrared (IR) spectroscopy, for example by comparing the intensity of a crosslinker signal in the open or cleaved state to the intensity of the crosslinker signal in crosslinked state. Additionally, or alternatively, the amount of open space and/or cross-link density within a hydrogel can be measured by viscosity, for example by comparing the cleaved or open polymer viscosity to fully crosslinked polymer viscosity. Additionally, or alternatively, the amount of open space and/or cross-link density within a hydrogel can be measured by comparing the hydrogel's molecular weight in the open or cleaved state to the molecular weight in the crosslinked state.

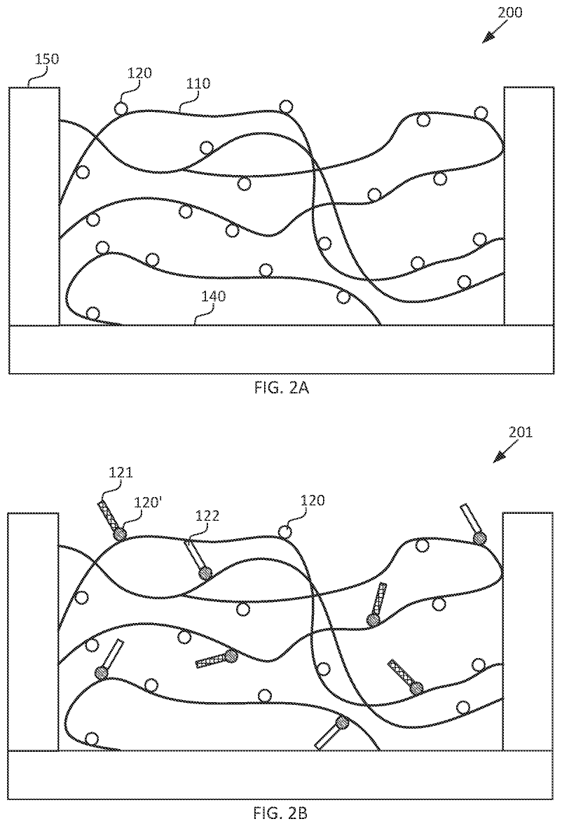

Although FIGS. 1A-1F describe the use of first functional groups 120 and second functional groups 130 that are of different types than one another, it should be appreciated that the first and second functional groups instead may be of the same type as one another. For example, FIGS. 2A-2C schematically illustrate alternative example compositions and operations in a process flow for using reversibly cross-linked hydrogels for cluster amplification. As illustrated in FIG. 2A, hydrogel 200 may be deposited on a substrate 140 in a similar manner as described with reference to FIG. 1A. Hydrogel 200 may include a three-dimensional network of polymer chains 110 and first functional groups 120 coupled to the polymer chains. In a similar manner as described with reference to FIG. 1B, amplification primers 121, 122 may be coupled to first functional groups 120 of the deposited hydrogel. However, as illustrated in FIG. 2B, the amplification primers 121, 122 may be coupled to a first subset of the first functional groups 120 of the deposited hydrogel (such functional groups being represented with darkened shading and reference numeral 120′ to suggest their reaction). A second subset of the first functional groups 120 remains unreacted, resulting in modified hydrogel 210 illustrated in FIG. 2B. The second subset of the first functional groups 120 of hydrogel 201 then may be converted to second functional groups 130, e.g., in a manner such as illustrated in FIG. 2C. For example, one or more suitable chemical or enzymatic reagents may be diffused into hydrogel 201 to convert the second subset of the first functional groups 120 into second functional groups 130, resulting in hydrogel 202 illustrated in FIG. 2C, which may be configured similarly as hydrogel 101 described with reference to FIG. 1B. Hydrogel 202 then may be stabilized by reversibly cross-linking the second functional groups 130, e.g., in a manner such as described with reference to FIG. 1C. The resulting reversibly cross-linked hydrogel then may be used in a manner such as described with reference to FIGS. 1D-1F.

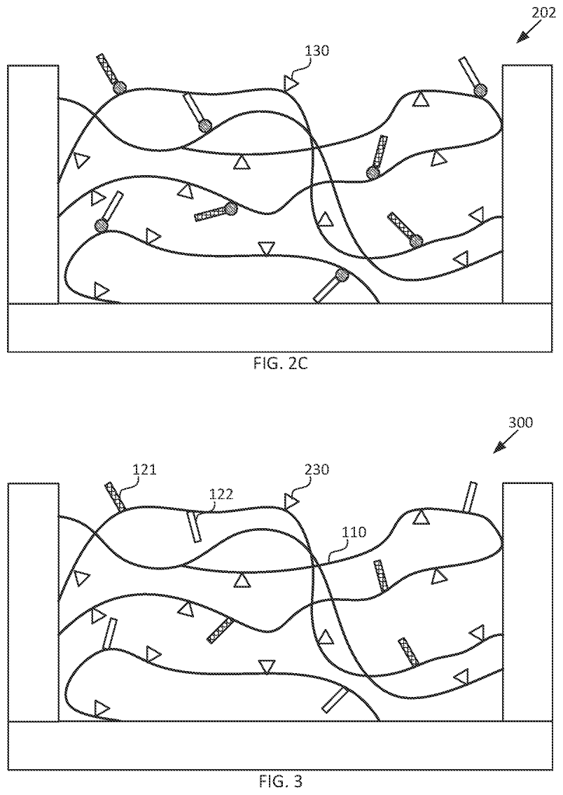

In another example, the hydrogel may be formed so as to include the amplification primers. For example, FIG. 3 schematically illustrates another alternative example composition and operation in a process flow for using reversibly cross-linked hydrogels for cluster amplification. In the example shown in FIG. 3, hydrogel 300 may be deposited on substrate 140 in a similar manner as described with reference to FIGS. 1A and 2B. Hydrogel 300 may include a three-dimensional network of polymer chains 110 similarly as hydrogels 100 and 200. Hydrogel 300 also may include amplification primers 121, 122 coupled to polymer chains 110. Illustratively, the amplification primers may be coupled to polymer chains 110 during preparation of hydrogel 300, e.g., before the hydrogel is deposited on substrate 140. Hydrogel 300 also may include functional groups 230 coupled to polymer chains 110 and configured similarly as second functional groups 130 such as described with reference to FIGS. 1A-1F and 2C. Hydrogel 300 may be stabilized by reversibly cross-linking the functional groups 230, e.g., in a manner such as described with reference to FIG. 1C. The resulting reversibly cross-linked hydrogel then may be used in a manner such as described with reference to FIGS. 1D-1F.

Note that the particular compositions, and the particular order of operations described with reference to FIGS. 1A-1F, 2A-2C, and 3 suitably may be modified in a variety of different ways. For example, the reversibly cross-linked hydrogel including amplification primers 121, 122 (e.g., hydrogel 102 described with reference to FIG. 1C) may be prepared by a manufacturer, and may be stable for storage and shipping to another entity, such as a customer. The entity may seed the polymer with target polynucleotides (e.g., to form hydrogel 103 described with reference to FIG. 1D). The entity then may reverse the cross-linkages 131 to open the hydrogel (e.g., to form hydrogel 104 described with reference to FIG. 1E). The entity then may amplify the seeded target polynucleotide within which the cross-linkages have been reversed to form a cluster (e.g., to form hydrogel 105 described with reference to FIG. 1F). The entity optionally may swell the hydrogel after reversing the cross-linkages and before the amplifying.

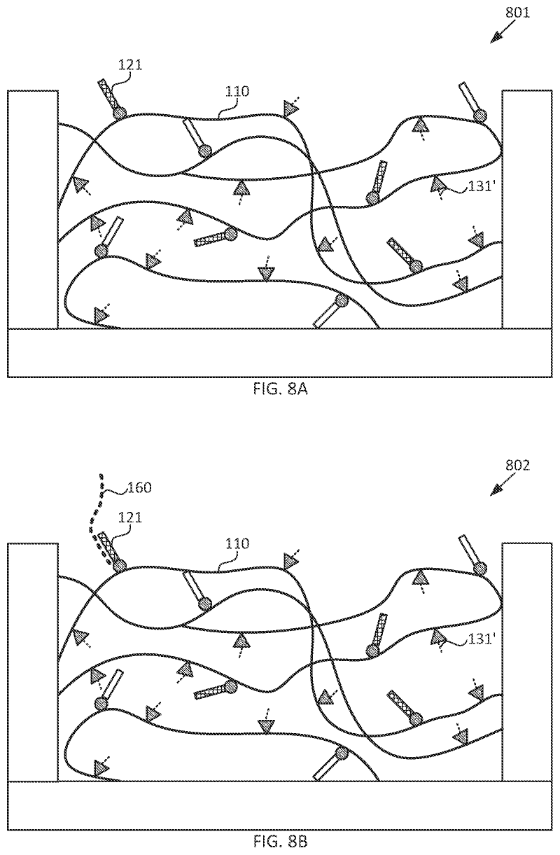

As noted further above, seeding need not necessarily be performed before reversing the hydrogel cross-linking. Illustratively, the hydrogel may be cross-linked after coupling the amplification primers, to provide cross-linked hydrogel 102 in a manner similar to that described with reference to FIGS. 1A-1C. Rather than seeding the cross-linked hydrogel 102 in a manner such as described with reference to FIG. 1D, in some examples the cross-linking within hydrogel 102 instead may be reversed prior to seeding. For example, FIGS. 8A-8B schematically illustrate additional example compositions and operations in a process flow for using reversibly cross-linked hydrogels for cluster amplification. Hydrogel 801 illustrated in FIG. 8A may be generated by cleaving the cross-linking within hydrogel 102 described with reference to FIG. 1C, in a manner such as described with reference to FIG. 1E, omitting an intervening operation of seeding the hydrogel as described with reference to FIG. 1D. After cleaving the cross-linking to form hydrogel 801 including amplification primers 121 and broken cross-linkages 131′, the hydrogel may be seeded with target polynucleotide 160 in a manner such as illustrated in FIG. 8B to form hydrogel 802 which is similar to hydrogel 104 described above, but obtained through different operations.

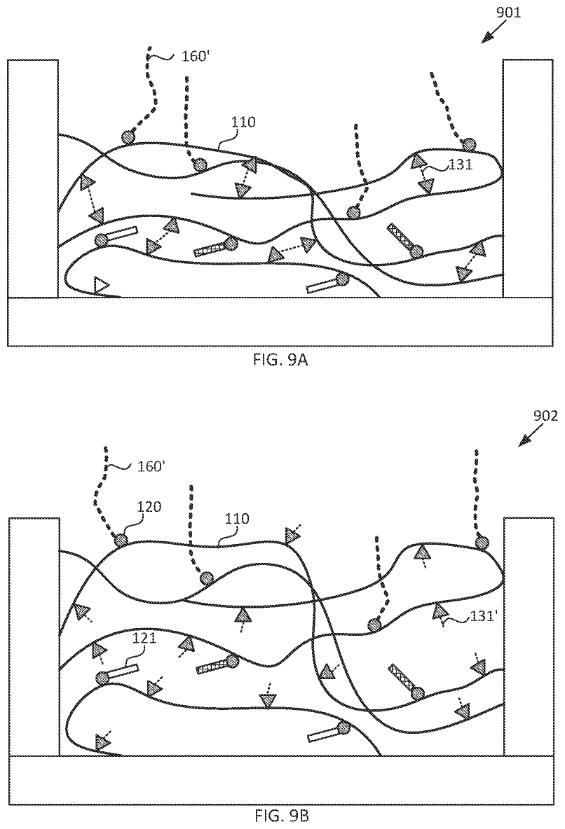

Additionally, or alternatively, as noted further above, reversing the cross-linking need not necessarily be performed at all, or completely, before amplification. Instead, amplification may be partially or fully performed before reversing the cross-linking. For example, FIGS. 9A-9B schematically illustrate additional example compositions and operations in a process flow for using reversibly cross-linked hydrogels for cluster amplification. Hydrogel 901 illustrated in FIG. 9A may be generated by performing cluster amplification in a manner similar to that described with reference to FIG. 1F, but using seeded, cross-linked hydrogel 103 described with reference to FIG. 1D. Note that hydrogel 901 may include significantly fewer amplicons 160′ than does hydrogel 105 described with reference to FIG. 1F, because significantly fewer amplification primers 121 are accessible to amplification reagents due to the cross-linking of hydrogel 901. For example, amplicons 160′ may be generated using amplification primers 121 that are sufficiently near to the outer surface of hydrogel 901 to be usable for cluster amplification, while other amplification primers 121 that are deeper within hydrogel 901 may remain inaccessible for cluster amplification due to the cross-linking. The cross-linkages within hydrogel 901 then may be cleaved in a manner such as described with reference to FIG. 1E, to provide hydrogel 902 (FIG. 9B) including amplification primers 121 and broken cross-linkages 131′. Optionally, amplicons 160′ may be further amplified using the newly accessible amplification primers 121 in a manner similar to that described with reference to FIG. 1F, to form hydrogel 105 through a different series of operations than described with reference to FIGS. 1E-1F.

Some nonlimiting examples of functional groups and their uses to form reversible cross-linkages within hydrogels now will be provided.

Any suitable functional groups may be included in a hydrogel and used to couple amplification primers 121, 122 to the hydrogel and/or to reversibly cross-link polymer chains 110 of the hydrogel to one another. In some examples, the functional groups (e.g., 120, 130, and/or 230) independently may be selected from the group consisting of: azide, amine, thiol, diol, aldehyde, alkyne, strained cyclooctyne, and an inverse electron-demand (IED) Diels-Alder group. The functional groups may be included in polymer chains 110 of the hydrogel 100, 200, or 300 during synthesis of the hydrogel. The cross-linking density may be controlled by tuning the content of the functional moieties 120, 130, and/or 230 in the polymer chains. Additionally, as noted above with reference to FIGS. 2A-2C, one type of functional group may be converted to another type of functional group within the hydrogel. In one nonlimiting example in which functional groups 120 include azides, a first set of the azides is used to couple amplification primers 121, 122 to polymer chains 110, and a second, unreacted set of the azides are reduced to amines which are used as functional groups 130 to cross-link polymer chains 110 to one another via a cross-linker molecule 131 that includes amine-reactive functional groups. Optionally, any suitable number of the azides may be coupled to heterobifunctional molecules that include one or more different types of azide-reactive functional groups (such as alkyne or DBCO), and also possess an orthogonal functional group (such as amine, thiol, diol, aldehyde or an inverse electron-demand (IED) Diels-Alder group). These functional groups may be used to crosslink the hydrogel using a cleavable molecule that reacts with them.

As noted above with reference to FIGS. 1A-1B, amplification primers 121, 122 may be coupled to functional groups that react with functional groups 120 in such a manner as to couple the amplification primers 121, 122 to polymer chains 110. In examples in which functional groups 120 are amines, nonlimiting examples of functional groups that may be coupled to amplification primers 121, 122 and that may react with the amines to couple the amplification primers to the polymer chains include: N-hydroxysuccinimide (NHS) ester, imidoester, pentofluorophenyl ester, hydroxymethyl phosphine, and carboxyl carbodiimide. In examples in which functional groups 120 are thiols, nonlimiting examples of functional groups that may be coupled to amplification primers 121, 122 and that may react with the thiols to couple the amplification primers to the polymer chains include: maleimide, haloacetyl, pyridyl disulfide, thiosulfonate, and vinyl sulfone. In examples in which functional groups 120 are aldehydes, nonlimiting examples of functional groups that may be coupled to amplification primers 121, 122 and that may react with the aldehydes to couple the amplification primers to the polymer chains include: hydrazide, alkoxyamine, and isocyanate. In examples in which functional groups 120 are azides, nonlimiting examples of functional groups that may be coupled to amplification primers 121, 122 and that may react with the azides to couple the amplification primers to the polymer chains include: alkynes, cyclooctynes, phosphines, and norbornenes. In examples in which functional groups are diols, a nonlimiting example of a functional group that may be coupled to amplification primers 121, 122 and that may react with the diols to couple the amplification primers to the polymer chains includes sulfonyl fluoride; the unreacted diols may be converted to dialdehydes which can be used to cross-link the polymer chains. In examples in which functional groups 120 are IED Diels-Alder groups, nonlimiting examples of IED Diels-Alder groups that may be coupled to amplification primers 121, 122 and that may react with the functional groups 120 to couple the amplification primers to the polymer chains include transcyclooctene or norbornene (which can react with functional group 120 that is tetrazine); tetrazine (which can react with functional group 120 that is transcyclooctene or norbornene). It will be appreciated that any combinations of the aforementioned functional groups may be used; for example, the functional groups coupled to the amplification primers may include amines, thiols, diols, aldehydes, or IED Diels-Alder group, and functional groups 120 may be selected to react with such functional groups in a similar manner as described above.

In a manner such as described with reference to FIGS. 1C-1E, 2C, and 3, functional groups 130, 230 may be used to reversibly cross-link the polymer chains in any suitable manner. In some examples, functional groups 130, 230 reversibly cross-link the polymer chains via cleavable molecules. For example, the cleavable molecules may form covalent bonds with functional groups 130, 230 that are coupled to different polymer chains 110 than one another, to thereby covalently cross-link those polymer chains to one another in a manner such as illustrated in FIG. 1C. Cleavable molecules may include, for example, aliphatic, aromatic, hydrophilic, or amphiphilic moieties. The cleavable molecules may be cleavable in any suitable manner, for example using a chemical agent, an enzyme, light, or heat. For example, the cleavable molecules may include at least one labile bond that may be cleaved upon application of a stimulus such as a chemical agent, enzyme, light, or heat.

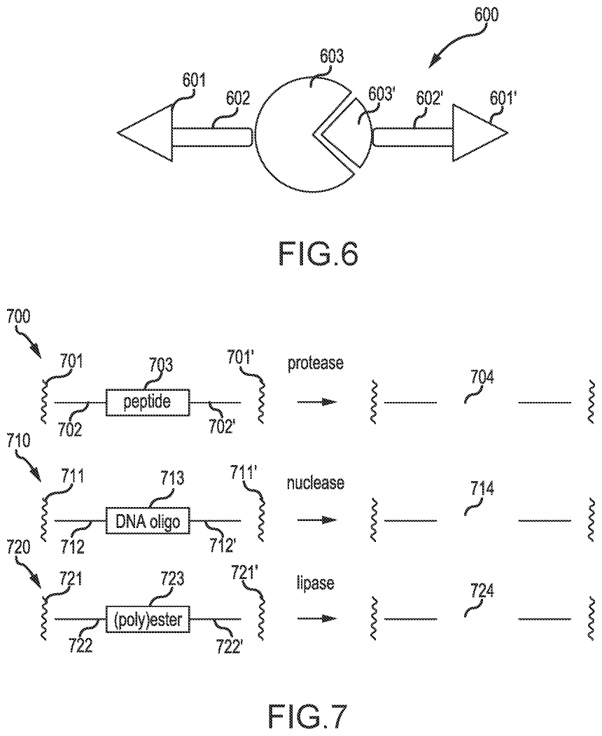

FIG. 6 schematically illustrates an example cleavable molecule 600 that may be used to reversibly cross-link hydrogels in a manner such as described herein. Molecule 600 includes first functional group 601, second functional group 601′, optional first linker 602, optional second linker 602′, first cleavable component 603, and second cleavable component 603′. First linker 602 may couple first functional group 601 to first cleavable component 603; alternatively, if first linker 602 is omitted, then first functional group 601 may be directly coupled to first cleavable component 603. Second linker 602 may couple second functional group 601′ to second cleavable component 603′; alternatively, if second linker 602′ is omitted, then second functional group 601′ may be directly coupled to second cleavable component 603′. First cleavable component 603 may be coupled to second cleavable component 603′ in a first, relatively closed state of the hydrogel, and may be decoupled from the second cleavable component in a second, relatively open state of the hydrogel. First functional group 601 may be coupled to a first one of functional groups 130 and second functional group 601′ may be coupled to a second one of functional groups 130, in a manner such as described with reference to FIG. 1C. As such, molecule 600 may couple a first polymer chain 110 (to which the first one of functional groups 130 is coupled) of hydrogel 101 to a second polymer chain 110 (to which the second one of functional groups 130 is coupled), causing the hydrogel to take on a relatively closed, more highly crosslinked configuration 102 such as described with reference to FIG. 1C.

Optional linkers 602, 602′ may have the same configuration as one another, or may have different configurations than one another. In some examples, one linker 602, 602′ is used, and

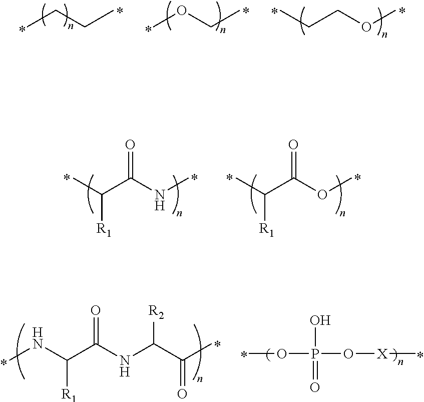

the other linker is omitted. Optional linkers 602, 602′ independently may include alkyl chains, polyethylene glycol (PEG), peptides, or polyphosphates, such as illustrated below: in which * denotes connection to the first or second functional group 601 or 601′ or to the first or second cleavable component 603, 603′, R₁ and R₂ denote residues of natural or non-natural amino acids, and X denotes a natural nucleoside or a spacer such as PEG or alkyl. In nonlimiting examples, n may be in the range of about 1 to 100, or about 2 to about 80, or about 5 to about 50, or about 2 to about 16.

Referring still to FIG. 6, first cleavable component 603 and second cleavable component 603′ may correspond to any components of a molecule that may be coupled to one another at a first time, and may be decoupled from one another at a second, different time responsive to exposure to a suitable stimulus. For example, first cleavable component 603 and second cleavable component 603′ may be covalently bonded to one another at a first time, e.g., while the hydrogel is in a relatively closed state such as described with reference to FIGS. 1C and 1D, and the covalent bond may be dissociated at a second time, e.g., to reopen the hydrogel in a manner such as described with reference to FIG. 1E. Table 1 below lists different types of chemical bonds that may be used to couple first cleavable component to second cleavable component 603′, as well as different categories of stimuli that may be used to dissociate such bonds (e.g., chemical, enzymatic, or photolabile stimuli) and specific examples of such stimuli.

| Category | Bond | Stimulus |
|---|---|---|
| Chemical | Vicinal Diols | Periodate |
| | Disulfides, azidoalkyl ether | Reducing agents TCEP etc. |
| | Acetals, ketals, imines | Acidic pH |
| | Allyl ether | Palladium/THP |
| Enzymatic | Oligonucleotides | Restriction enzymes |
| | Peptides | Protease |
| | Ester linkage | Lipases |
| | Saccharides | Cellulase |
| Photolabile | o-Nitrophenyl | UV irradiation |

Nonlimiting examples of cleavable components with covalent bonds that may be dissociated using chemical stimuli such as listed in Table 1 are shown below:

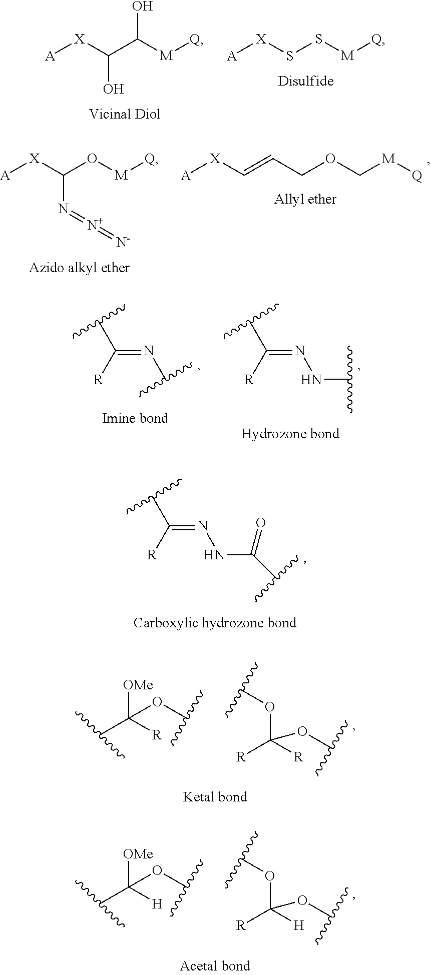

in which A and Q (if shown) correspond to first or second functional group 601 or 601′, X and M (if shown) correspond to first or second linker 602, 602′, where the squiggly lines (if shown) correspond to the first or second functional group 601 or 601′ or to the first or second cleavable component 603, 603′, and where R corresponds to a functional group that includes one or more carbon atoms and optionally includes one or more heteroatoms, such as alkyl, allyl, or aryl. For further details regarding chemically cleavable linkers, see Leriche et al., “Cleavable linkers in chemical biology,” Bioorg. Med. Chem. 20(2): 571-782 (2012), the entire contents of which are incorporated by reference herein.

Nonlimiting examples of cleavable components with a covalent bond that may be reversibly associated and disassociated using thermal stimuli are shown below:

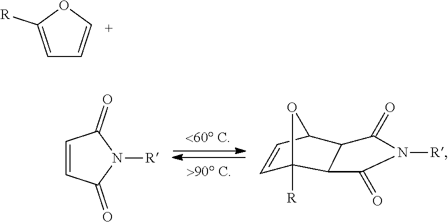

in which R and R′ are the same as one another or may differ from one another, and include functional groups that are reactive with functional groups on the polymer in a manner such as described elsewhere herein.

In some examples in which the cleavable molecules are cleavable by a chemical agent, the chemical agent may include an acid. Nonlimiting examples of labile moieties include an acetal, ketal, imine, hydrazone, or t-butyl ester including a bond that is cleavable by the acid, e.g., by use of a buffer with a pH that is about 5 or less. An example cleavable molecule is illustrated below, in which the ketal moiety may be replaced by any of the alternative labile moieties illustrated below the molecule:

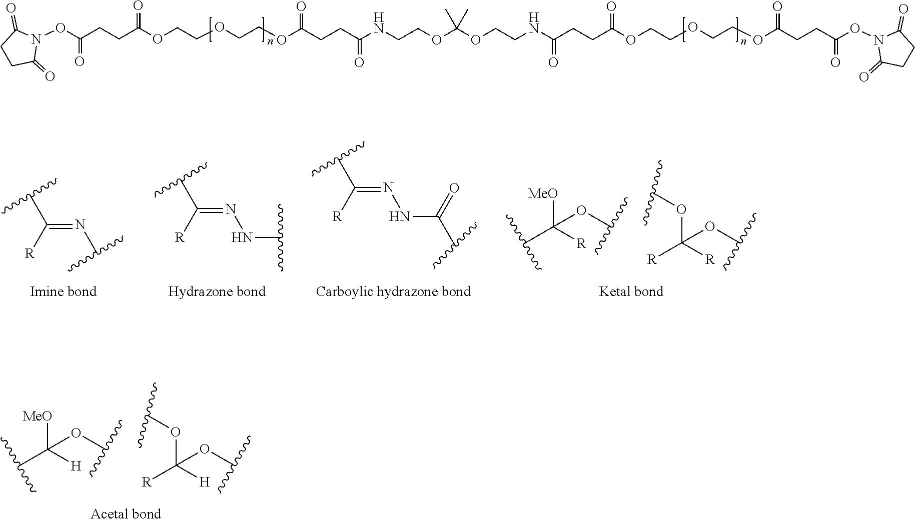

For further details regarding pH sensitive linkers including imine, hydrazine, or acetal moieties, see Myrgorodska et al., “A novel acid-degradable PEG cross-linker for the fabrication of pH-responsive soft materials,” Macro-Molecular Rapid Communications 42(12): 2100102 (2021), the entire contents of which are incorporated by reference herein.

In one specific, nonlimiting example, the acid may include periodic acid, sodium periodate, or lead tetraacetate. The cleavable molecules may include a vicinal diol that is cleavable by the periodic acid. A nonlimiting example of a cleavable molecule that includes a vicinal diol cleavable by periodic acid is:

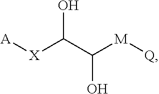

in which A and Q correspond to first or second functional group 601 or 601′, and X and M correspond to first or second linker 602, 602′.

In other examples in which the cleavable molecules are cleavable by a chemical agent, the chemical agent may include a reducing agent. A nonlimiting example of a labile bond is a disulfide bond that is cleavable using the reducing agent. Another nonlimiting example of a labile bond is an azidoalkyl ether that is cleavable using the reducing reagent. Another nonlimiting example of a labile bond is an allyl ether that is cleavable using a palladium complex of the reducing agent. Illustratively, the reducing agent may include glutathione, dithiothreitol (DTT), beta-mercaptoethanol (BME), cystamine, or tris(2-carboxyethyl)phosphine (TCEP). A nonlimiting example of a cleavable molecule with a disulfide bond that is cleavable using a reducing agent is:

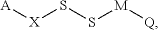

in which A and Q correspond to first or second functional group 601 or 601′, and X and M correspond to first or second linker 602, 602′. A nonlimiting example of a cleavable molecule with an azidoalkyl ether that is cleavable using a reducing agent is:

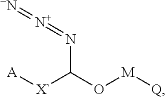

in which A and Q correspond to first or second functional group 601 or 601′, and X and M correspond to first or second linker 602, 602′.

FIG. 7 schematically illustrates additional example cleavable molecules 701, 711, 721 that may be used to reversibly cross-link hydrogels in a manner such as described herein. Molecule 700 includes first functional group 701, second functional group 701′, first linker 702, second linker 702′, and cleavable component(s) 703 which may include a first cleavable component and second cleavable component in a manner similar to that described with reference to FIG. 6. First linker 702 may couple first functional group 701 to cleavable component(s) 703; alternatively, if first linker 702 is omitted, then first functional group 701 may be directly coupled to cleavable component(s) 703. Second linker 702′ may couple second functional group 701′ to cleavable component(s) 703; alternatively, if second linker 702′ is omitted, then second functional group 701′ may be directly coupled to cleavable component(s) 703. Cleavable components may be coupled to one another in a first, relatively closed state of the hydrogel, and may be decoupled from one another and/or may be fully or partially removed from molecule 701 in a second, relatively open state of the hydrogel. First functional group 701 may be coupled to a first one of functional groups 130 and second functional group 701′ may be coupled to a second one of functional groups 130, in a manner such as described with reference to FIG. 1C. As such, molecule 700 may couple a first polymer chain 110 (to which the first one of functional groups 130 is coupled) of hydrogel 101 to a second polymer chain 110 (to which the second one of functional groups 130 is coupled), causing the hydrogel to take on a relatively closed, more highly crosslinked configuration 102 such as described with reference to FIG. 1C. In the nonlimiting example shown in FIG. 7, cleavable component(s) 703 include a polypeptide that may be fully or partially degraded using a protease to generate gap 704 that causes the hydrogel to reopen in a manner such as described with reference to FIG. 1E. Note that such a protease may be nonspecific to the polypeptide. Alternatively, cleavable component 703 may include a specific peptide sequence that is recognized and specifically cleaved by a lysosomal enzyme.

Molecule 710 illustrated in FIG. 7 includes first functional group 711, second functional group 711′, first linker 712, second linker 712′, and cleavable component(s) 713 which may include a first cleavable component and second cleavable component in a manner similar to that described with reference to FIG. 6. First linker 712 may couple first functional group 711 to cleavable component(s) 713; alternatively, if first linker 712 is omitted, then first functional group 711 may be directly coupled to cleavable component(s) 713. Second linker 712′ may couple second functional group 711′ to cleavable component(s) 713; alternatively, if second linker 712′ is omitted, then second functional group 711′ may be directly coupled to cleavable component(s) 713. Cleavable components may be coupled to one another in a first, relatively closed state of the hydrogel, and may be decoupled from one another and/or may be fully or partially removed from molecule 711 in a second, relatively open state of the hydrogel. First functional group 711 may be coupled to a first one of functional groups 130 and second functional group 711′ may be coupled to a second one of functional groups 130, in a manner such as described with reference to FIG. 1C. As such, molecule 710 may couple a first polymer chain 110 (to which the first one of functional groups 130 is coupled) of hydrogel 101 to a second polymer chain 110 (to which the second one of functional groups 130 is coupled), causing the hydrogel to take on a relatively closed, more highly crosslinked configuration 102 such as described with reference to FIG. 1C. In the nonlimiting example shown in FIG. 7, cleavable component(s) 713 include an oligonucleotide that may be fully or partially degraded using a nuclease (such as a DNAase in examples in which the oligonucleotide includes DNA, or an RNAase in which the oligonucleotide includes RNA, or a protease in examples in which the oligonucleotide includes peptide nucleic acid (PNA)) to generate gap 714 that causes the hydrogel to reopen in a manner such as described with reference to FIG. 1E. Note that a DNAase may be nonspecific to the DNA oligonucleotide, an RNAase may be nonspecific to the RNA oligonucleotide, or a protease may be nonspecific to the PNA oligonucleotide. Alternatively, cleavable component 703 may include a specific oligonucleotide sequence that is recognized and specifically cleaved by a restriction enzyme.

Molecule 720 illustrated in FIG. 7 includes first functional group 721, second functional group 721′, first linker 722, second linker 722′, and cleavable component(s) 723 which may include a first cleavable component and second cleavable component in a manner similar to that described with reference to FIG. 6. First linker 722 may couple first functional group 721 to cleavable component(s) 723; alternatively, if first linker 722 is omitted, then first functional group 721 may be directly coupled to cleavable component(s) 723. Second linker 722′ may couple second functional group 721′ to cleavable component(s) 723; alternatively, if second linker 722′ is omitted, then second functional group 721′ may be directly coupled to cleavable component(s) 723′. Cleavable components may be coupled to one another in a first, relatively closed state of the hydrogel, and may be decoupled from one another and/or may be fully or partially removed from molecule 721 in a second, relatively open state of the hydrogel. First functional group 721 may be coupled to a first one of functional groups 130 and second functional group 721′ may be coupled to a second one of functional groups 130, in a manner such as described with reference to FIG. 1C. As such, molecule 720 may couple a first polymer chain 110 (to which the first one of functional groups 130 is coupled) of hydrogel 101 to a second polymer chain 110 (to which the second one of functional groups 130 is coupled), causing the hydrogel to take on a relatively closed, more highly crosslinked configuration 102 such as described with reference to FIG. 1C. In the nonlimiting example shown in FIG. 7, cleavable component(s) 723 include polymer that may be fully or partially degraded to generate gap 724 that causes the hydrogel to reopen in a manner such as described with reference to FIG. 1E. In some examples, the polymer may include a (poly)ester that may be fully or partially degraded using a lipase to generate gap 724. In other examples, the polymer may include an ester that includes a cross-linker which may be cleaved in a manner such as described herein, or an ester-containing oligomer/polymer that may be cleaved in a manner such as described herein.

Functional groups 701, 701′, 711, 711′, and 721, 721′ may have the same configuration as one another, or may have different configurations than one another, and in some examples may be selected from the options provided above for functional groups 601, 601′. Linkers 702, 702′, 712, 712′, and 722, 722′ may have the same configuration as one another, or may have different configurations than one another, and in some examples may be selected from the options provided above for linkers 602, 602′.

In some examples in which the cleavable molecules are cleavable by an enzyme, the enzyme may include a restriction enzyme, and the cleavable molecules may include an oligonucleotide that is cleavable using the restriction enzyme in a manner such as described with reference to molecule 710 of FIG. 7. The oligonucleotide may include a functional group on each end that can form a covalent bond with functional groups 130, 230 to reversibly cross-link polymer chains 110 with one another. In other examples in which the cleavable molecules are cleavable by an enzyme, the enzyme may include a protease enzyme, and the cleavable molecules may include a peptide that is cleavable using the protease enzyme in a manner such as described with reference to molecule 700 of FIG. 7. The peptide may include a functional group on each end that can form a covalent bond with functional groups 130, 230 to reversibly cross-link polymer chains 110 with one another. Nonlimiting examples of commercially available peptides that may be used as cleavable component(s) 703 and are cleavable using proteases are shown below:

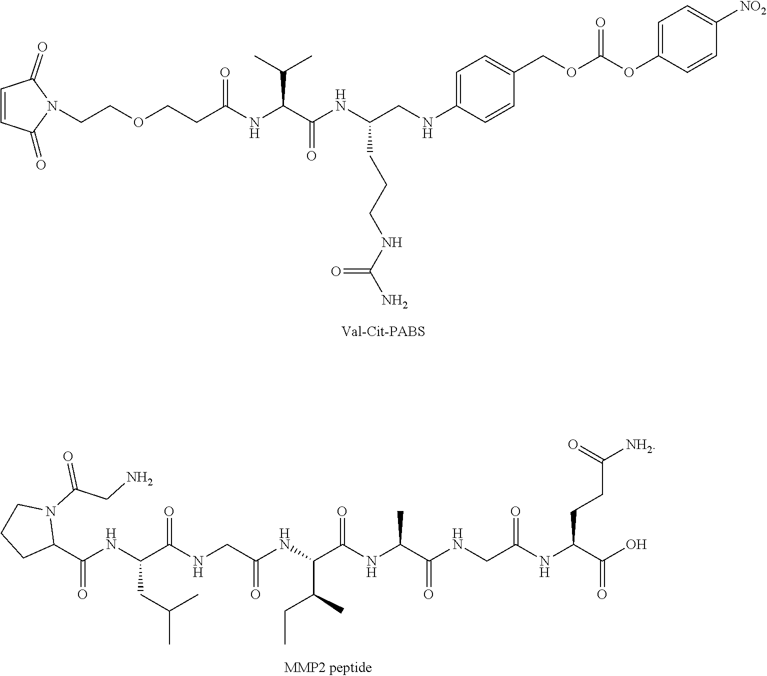 [SMILES: CC(C)[C@H](NC(=O)CCOCCN1C(=O)C=CC1=O)C(=O)N[C@@H](CCCNC(N)=O)CNc1ccc(COC(=O)Oc2ccc([N+](=O)[O-])cc2)cc1 ; CC[C@H](C)[C@H](NC(=O)CNC(=O)[C@H](CC(C)C)NC(=O)C1CCCN1C(=O)CN)C(=O)N[C@@H](C)C(=O)NCC(=O)N[C@@H](CCC(N)=O)C(=O)O]

In some examples, Val-Cit-PABS (valine-citrulline-p-aminobenzyl alcohol) may be coupled to functional groups, such as maleimide and/or p-nitrophenyl carbonate, for cross-linking. Another example of enzyme-cleavable component(s) 703 is matrix metalloproteinase-2 cleavable peptide (MMP2), which may be functionalized by reactive groups for cross-linking as well. As provided elsewhere herein, the functional groups of the present cross-linkers may be identical to one another, or may be different than one another.

In examples in which the cleavable molecules are cleavable by light, the cleavable molecules may include a coumarin or nitrobenzene group that is cleavable using light. A nonlimiting example of a molecule that includes a nitrobenzene group that is cleavable using light of about 360 nm is:

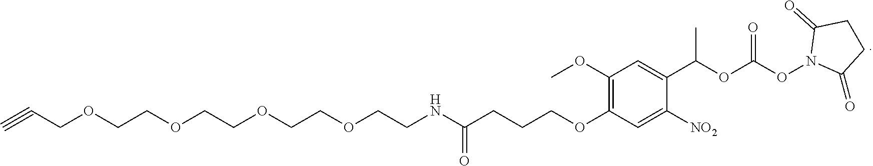 [SMILES: C#CCOCCOCCOCCOCCNC(=O)CCCOc1cc([N+](=O)[O-])c(C(C)OC(=O)ON2C(=O)CCC2=O)cc1OC]

For further details regarding molecules that may be cleaved using light (optionally in combination with a chemical reagent), see Leriche et al., cited above, as well as Hansen et al., “Wavelength-selected cleavage of photoprotecting groups: strategies and applications in dynamic systems,” Chemical Society Reviews 44: 3358-3377 (2015), the entire contents of which are incorporated by reference herein.

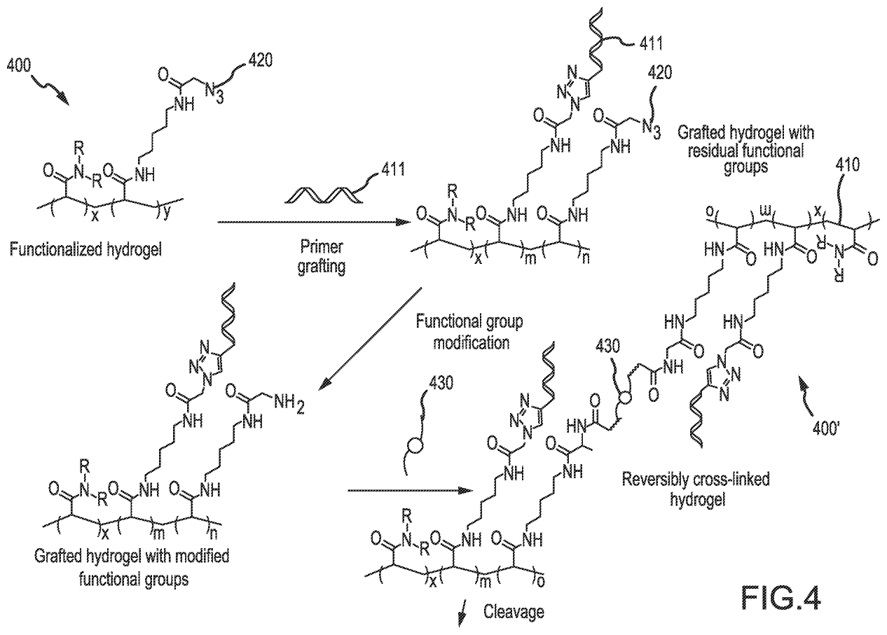

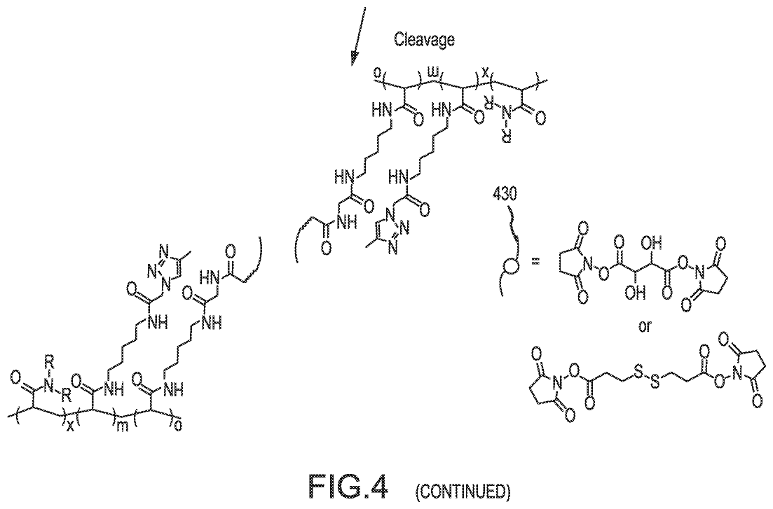

FIG. 4 schematically illustrate example hydrogels and example cross-linkers that may be used with such hydrogels. Example hydrogel 400 illustrated in FIG. 4 includes poly(N-(5-azidoacetamidylpentyl)acrylamide-co-acrylamide) (PAZAM) such as described in International Patent Publication No. WO2013/184796, the entire contents of which are incorporated by reference herein. In the nonlimiting example illustrated in FIG. 4, a first subset of functional groups 420 (e.g., azide groups) of hydrogel 400 are coupled to oligonucleotides 411, e.g., amplification primers in a manner such as described with reference to FIG. 1B, via “primer grafting” such as described with reference to FIG. 1B. A second subset of the functional groups 420 (e.g., azide groups) are not coupled to amplification primers, and thus may be considered “residual.” Such residual functional groups are available for use in reversibly cross-linking the hydrogel. The residual functional groups optionally may be modified to form another type of functional group. For example, in the nonlimiting example shown in FIG. 4, the second subset of the azide groups may be contacted with a reducing agent, such as a phosphine, which reduces the azide groups to amines. The grafted hydrogel with modified functional groups then is contacted with a reversibly cleavable molecule 430 (such as described in greater detail above, some nonlimiting examples of which are shown in FIG. 4). In the nonlimiting example shown in FIG. 4, the functional groups of the cleavable molecule 430 (e.g., NHS groups) react with the amines resulting from reaction of the azides with the reducing agent, thus forming reversibly cross-linked hydrogel 400′ including polymer chains 110 that are cross-linked using reacted cleavable molecules 431. At a later time (e.g., before or after seeding and/or amplification), hydrogel 400′ may be contacted with a suitable stimulus to cleave cleavable molecules 431, allowing the hydrogel to reopen in a manner such as described with reference to FIG. 1E.

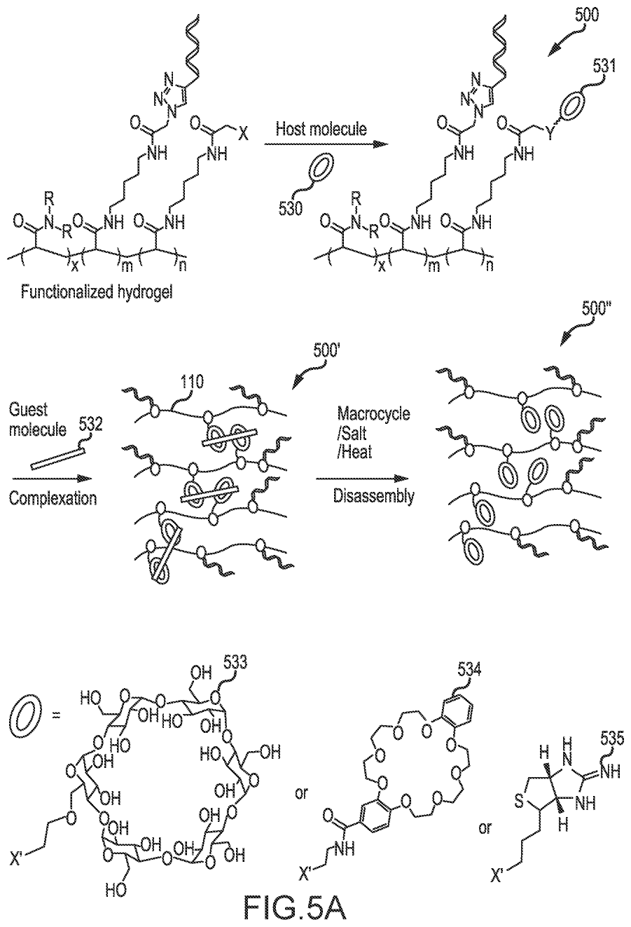

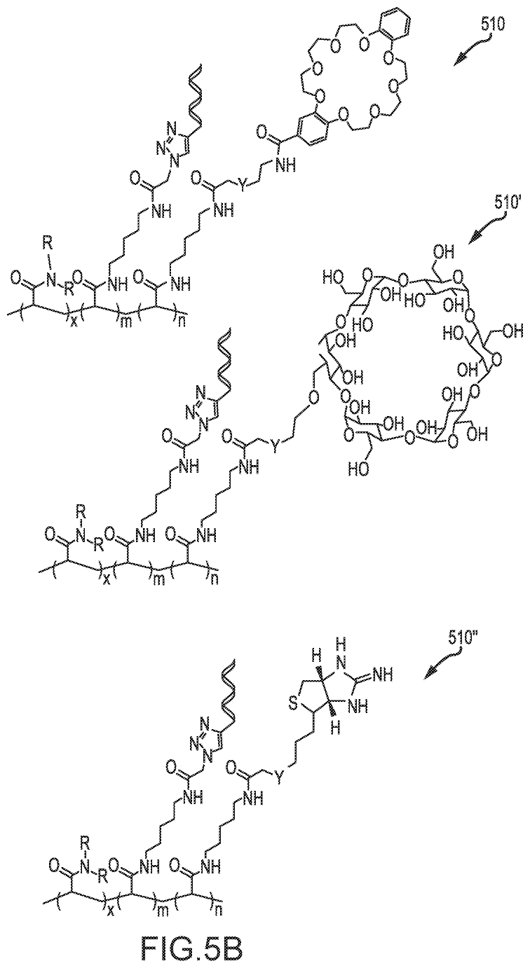

As noted elsewhere herein, noncovalent interactions alternatively may be used to cross-link polymer chains 110 within the hydrogel. Illustratively, functional groups 130, 230 may include host molecules that reversibly cross-link the backbone via guest molecules. The guest molecules may be removable via salt, heat, or pH. Alternatively, the guest molecules may be removable via displacement with a binding partner to the guest molecules. For example, FIGS. 5A-5B schematically illustrate additional example hydrogels and example cross-linkers that may be used with such hydrogels. Hydrogel 500 illustrated in FIG. 5A may be prepared using operations that include reacting the functional groups of hydrogel 400 from FIG. 4 with host molecules 530. As illustrated in FIG. 5A, the functionalized hydrogel may include functional groups X, which are reactive with functional group X′ of host molecules 530. X and X′ may include any suitable reactive pairs of functional groups, nonlimiting examples of which are provided elsewhere herein. Hydrogel is contacted with guest molecules 532 such as illustrated in FIG. 5A. In a complexation process, the guest molecules 532 non-covalently bond to reacted host molecule moieties 531 that are coupled to different polymer chains 110 than one another, thus providing hydrogel 500′ including cross-linked polymer chains. In a disassembly process, the non-covalent bonds between the guest molecules 532 and host molecule moieties 531 are broken (e.g., using salt, heat, or pH) causing reversal of the cross-linkages that had been formed using the complexation process. In some examples, host molecule moieties 531 include macrocyclic pendant groups 533 or 534, and guest molecules 531 suitably are selected for use with such macrocyclic pendant groups. A variety of host-guest partners and their binding affinities have been reported in literature. Nonlimiting examples of hydrogels 510 and 510′ are illustrated in FIG. 5B that may be formed respectively using pending groups 533 or 534 illustrated in FIG. 5A. Hydrogel 510 includes crown ethers as host molecule moieties 531, which may be used with guest molecules 532 that include terminal ammonium moieties coupled to a linker such as described elsewhere herein. Hydrogel 510′ includes beta-cyclodextrins as host molecule moieties 531, which may be used with guest molecules 532 such as adamantanes, ferrocenes, or bipyridines.

In still other examples, the functional groups of the hydrogel may be coupled to ligand molecules that reversibly cross-link the backbone via multivalent binding proteins. The multivalent binding proteins are removable using a denaturing agent. For example, protein-ligand complexes held by strong non-covalent interactions may be employed. In a manner similar to that described with reference to macrocycle-guest binding, hydrogel 500 may include ligand molecules 535 in their backbone as illustrated in FIG. 5A. Incubation with an appropriate multivalent binding protein can induce crosslinking among chains which can be reversed by displacing the protein with excess free ligands or harsh chaotropic agents. One example of this is biotin-streptavidin linkages, where the streptavidin binds up to 4 biotin molecules 535. Another example is binding between concanavalin A protein and four specific sugars, e.g., D-glucose and D-mannose. Hydrogel 510″ illustrated in FIG. 5B includes a ligand 535, a nonlimiting example of which is illustrated in FIG. 5A, and which may be cross-linked using a multi-valent protein. For example, the strong but reversible interaction between certain proteins and their small molecule binding partners may be used to cross-link hydrogel 510″. The small molecule ligands 535 bind in specific pockets of the protein via non-covalent interactions like H-bonding, salt bridges, or the like. When a protein is introduced to a polymer network with ligand side chains 535, the protein may bind to ligands from two or more distinct polymer chains, leading to a crosslinked hydrogel. The crosslinking can be reversed by introducing excess free-floating ligand to outcompete the binding of the polymer side chains.

Additional comments

It will be appreciated that the present compositions and methods may be used in any suitable application, such as amplifying a template polynucleotide. For example, although the present compositions may be used to perform “bridge amplification” or “surface-bound polymerase chain reaction,” it will be appreciated that the present compositions and methods readily may be adapted for use with other amplification modalities. One such amplification modality is “exclusion amplification,” or ExAmp. Exclusion amplification methods may allow for the amplification of a single target polynucleotide per region of the hydrogel and the production of a substantially monoclonal population of amplicons in that region. For example, the rate of amplification of the first seeded target polynucleotide within a region of the hydrogel may be more rapid relative to much slower rates of transport and seeding of target polynucleotides in that region. As such, the first target polynucleotide that seeds in a hydrogel region may be amplified rapidly and fill the entire hydrogel region, thus inhibiting the seeding of additional target polynucleotide(s) in the same hydrogel region. Alternatively, if a second target polynucleotide attaches to same region of the hydrogel after the first polynucleotide, the relatively rapid amplification of the first target polynucleotide may fill enough of the region to result in a signal that is sufficiently strong to perform sequencing by synthesis (e.g., the region may be at least functionally monoclonal). The use of exclusion amplification may also result in super-Poisson distributions of monoclonal regions; that is, the fraction of regions in an array that are functionally monoclonal may exceed the fraction predicted by the Poisson distribution.

Increasing super-Poisson distributions of useful clusters is useful because more functionally monoclonal regions may result in higher quality signal, and thus improved SBS; however, the seeding of target polynucleotides into regions of the hydrogel may follow a spatial Poisson distribution, where the trade-off for increasing the number of occupied regions is increasing the number of polyclonal regions. One method of obtaining higher super-Poisson distributions is to have seeding occur quickly, followed by a delay among the seeded target polynucleotide. The delay, termed “kinetic delay” because it is thought to arise through the biochemical reaction kinetics, gives one seeded target polynucleotide an earlier start over the other seeded targets. Exclusion amplification works by using recombinase to facilitate the invasion of primers (e.g., primers attached to a substrate region) into double-stranded DNA (e.g., a target polynucleotide) when the recombinase mediates a sequence match. The present compositions and methods may be adapted for use with recombinase to facilitate the invasion of the present amplification primers and orthogonal amplification primers into the present target polynucleotides when the recombinase mediates a sequence match. Indeed, the present compositions and methods may be adapted for use with any surface-based polynucleotide amplification methods such as thermal PCR, chemically denatured PCR, and enzymatically mediated methods (which may also be referred to as recombinase polymerase amplification (RPA) or ExAmp).

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A hydrogel on a substrate, comprising:
a three-dimensional network of polymer chains;
first functional groups coupled to the polymer chains;
amplification primers coupled to the polymer chains via the first functional groups; and
second functional groups coupled to the polymer chains and reversibly cross-linking the polymer chains to one another.

2. The hydrogel of claim 1, wherein the first and second functional groups are of different types than one another.

3. The hydrogel of claim 1, wherein the first and second functional groups are of the same type as one another.

4. The hydrogel of claim 1, wherein the first and second functional groups independently are selected from the group consisting of: azide, amine, thiol, diol, aldehyde, alkyne, strained cyclooctyne, and an inverse electron-demand (IED) Diels-Alder group.

5. The hydrogel of claim 1, wherein the second functional groups reversibly cross-link the polymer chains via cleavable molecules.

6. The hydrogel of claim 5, wherein the cleavable molecules are cleavable using a chemical agent, an enzyme, light, or heat.

7. The hydrogel of claim 6, wherein the chemical agent comprises an acid.

8. The hydrogel of claim 7, wherein the cleavable molecules comprise an acetal, ketal, imine, hydrazone, or t-butyl ester that is cleavable by the acid.

9. The hydrogel of claim 6, wherein the chemical agent comprises a reducing agent.

10. The hydrogel of claim 8, wherein the cleavable molecules comprise a disulfide bond or azidoalkyl ether that is cleavable using the reducing agent, or allyl ether that is cleavable using a palladium complex of the reducing agent.

11. The hydrogel of claim 6, wherein the enzyme comprises a DNAase, RNAase, protease, or restriction enzyme, and wherein the cleavable molecules comprise an oligonucleotide that is cleavable using the DNAase, RNAase, protease, or restriction enzyme.

12. The hydrogel of claim 6, wherein the enzyme comprises a protease enzyme or lysosomal enzyme, and wherein the cleavable molecules comprise a peptide that is cleavable using the protease enzyme or lysosomal enzyme.

13. The hydrogel of claim 6, wherein the cleavable molecules comprise a Diels-Alder conjugation that is cleavable using heat.

14. The hydrogel of claim 6, wherein the cleavable molecules comprise a coumarin or nitrobenzene group that is cleavable using light.

15. The hydrogel of claim 1, wherein the second functional groups comprise host molecules that reversibly cross-link the backbone via guest molecules.

16. The hydrogel of claim 15, wherein the guest molecules are removable via salt, heat, or pH.

17. The hydrogel of claim 15, wherein the guest molecules are removable via displacement with a binding partner to the guest molecules.

18. The hydrogel of claim 15, wherein the host molecules comprise crown ethers and the guest molecules comprise ammonium moieties.

19. The hydrogel of claim 15, wherein the host molecules comprise beta-cyclodextrins and the guest molecules comprise adamantanes, ferrocenes, or bipyridines.

20. The hydrogel of claim 1, wherein the second functional groups comprise ligand molecules that reversibly cross-link the backbone via multivalent binding proteins.

21. The hydrogel of claim 20, wherein the multivalent binding proteins are removable using a denaturing agent.

22. A method of using a hydrogel, the method comprising:
depositing a hydrogel on a substrate, the hydrogel comprising three-dimensional network of polymer chains and at least first and second types of functional groups coupled to the polymer chains;
coupling amplification primers to the first functional groups of the deposited hydrogel; and
reversibly stabilizing the deposited hydrogel by reversibly cross-linking the second functional groups of the deposited hydrogel to which the amplification primers are coupled.

23. A method of using a hydrogel, the method comprising:
depositing a hydrogel on a substrate, the hydrogel comprising a three-dimensional network of polymer chains, amplification primers coupled to the polymer chains, and functional groups coupled to the polymer chains; and
reversibly stabilizing the hydrogel by reversibly cross-linking the functional groups of the deposited hydrogel to which the amplification primers are coupled.

24. A method of using a hydrogel, the method comprising:
depositing a hydrogel on a substrate, the hydrogel comprising three-dimensional network of polymer chains and first functional groups coupled to the polymer chains;
coupling amplification primers to a first subset of the first functional groups of the deposited hydrogel;
converting a second subset of the first functional groups to second functional groups; and
reversibly stabilizing the hydrogel by reversibly cross-linking the second functional groups.

25. A method of using a hydrogel, the method comprising:
hybridizing a target polynucleotide to an amplification primer coupled to a hydrogel;
cleaving cross-linkages within the hydrogel within which the target polynucleotide is hybridized to the amplification primer; and
amplifying the target polynucleotide using additional amplification primers within the hydrogel within which the cross-linkages have been cleaved.

26. The method of claim 25, further comprising swelling the hydrogel after the cleaving and before the amplifying.
